(19) United States
(12) Patent Application Publication
(10) Pub. No.: US 2020/0100535 A1
(43) Pub. Date: Apr. 2, 2020

(54) DIETARY SUPPLEMENT FOR LOWERING BLOOD SUGAR, INCREASING IMMUNITY, AND INCREASING CARDIAC FUNCTION AND INCREASING PHYSICAL STAMINA

(71) Applicant: Ms.I-Szu Hsia, San Jose, CA (US)

(72) Inventors: I-Szu Hsia, San Jose, CA (US); Shuai Mao, Guandong (CN)

(21) Appl. No.: 16/590,141

(22) Filed: Oct. 1, 2019

Related U.S. Application Data
(60) Provisional application No. 62/739,772, filed on Oct. 1, 2018.

(51) Int. Cl. A23L 33/105; A23L 33/16; A61K 36/258; A61K 36/481; A61K 36/8969; A61K 36/718; A61K 36/428; A61K 36/488; A61K 35/644; A61K 33/24

(52) U.S. Cl. CPC A23L 33/105; A23L 33/16; A61K 36/258; A61K 36/481; A61K 33/24; A61K 36/718; A61K 36/428; A61K 36/488; A61K 35/644; A61K 36/8969

(57) ABSTRACT

This document provides dietary supplement compositions as well as methods for using dietary supplement compositions to lower blood sugar, increase immunity, improve cardiac function and increase physical stamina of humans or animals. An herbal dietary supplement composed of American Ginseng, Radix Astragali, Rhizoma Polygonati Odorati, and one or more of Rhizoma Coptidis, Radix Trichosanthis, Radix Puerariae Lobatae, Propolis, and chromium. The dietary supplement is effective as shown in various clinical trials to provide health benefits of lowering blood sugar, increasing immunity, improving cardiac function, supporting kidney health, and increasing physical stamina field of the invention.

### FIELD OF THE INVENTION

This invention relates to herbal compositions. More specifically, this invention relates to herbal compositions as dietary supplements that provide the humans or animals health benefits of lowering blood sugar, increasing immunity, improving cardiac function and increasing physical stamina of the invention.

### BACKGROUND OF THE INVENTION

A large number of consumers are seeking natural alternatives to synthetic pharmaceutical products to aid with a variety of ailments and to improve health, particularly chronic health issues such as high blood pressure and high blood sugar levels. The market has a great need to provide dietary supplements containing natural substances. Specifically, there are increased demands for herbal compositions as dietary supplements that provide the health benefits of lowering blood sugar, improving cardiac function, supporting kidney health, and reducing fatigue by increasing physical stamina.

### SUMMARY OF THE INVENTION

An object of this invention is to provide a more convenient and effective way to make and provide effective herbal compositions as dietary supplements that provide the humans or animals health benefits of lowering blood sugar, increasing immunity, improving heart function, supporting kidney health and reducing fatigue by increasing physical stamina.

Specifically, in a preferred embodiment the invention discloses a dietary supplement composed of American Ginseng, Radix Astragali, Rhizoma Polygonati Odorati, and one of the following: Rhizoma Coptidis, Radix Trichosanthis, Radix Puerariae Lobatae, Propolis, and chromium. The dietary supplement is effective as shown in various clinical trials to provide the health benefits of improving insulin sensitivity, lowering blood sugar, increasing immunity, improving cardiac function and increasing physical stamina field of the invention. A dietary supplement composition provided herein can be in the form of a beverage or a powder adapted to be mixed with water to form a beverage, liquid, soup, soup mix, solution, syrup, suspension, pill, powder, capsule, tablet, shake, bar, or other suitable ingestible carrier as is known in the art.

These and other objects and advantages of the present invention will no doubt become obvious to those of ordinary skill in the art after having read the following detailed description, which is illustrated in the various exemplary compositions and experimental clinic methods.

### BRIEF DESCRIPTION OF THE DRAWINGS

FIG. 1 shows the chemical composition of the dietary supplement extracted from Ginsenoside.

FIGS. 2 and 3 show the chemical compositions of the dietary supplements extracted from Radix Astragali.

### DETAILED DESCRIPTION OF THE INVENTION

The present invention is described in detail based on the preferred embodiments illustrated in various exemplary compositions and experimental clinic methods. Specifically, the present invention relates to an apparatus that comprises a dietary composition that comprises American Ginseng, Radix Astragali, Rhizoma Polygonati Odorati, and one or more ingredients selected from Rhizoma Coptidis, Radix Trichosanthis, Radix Puerariae Lobatae, Propolis, and chromium.

The composition contains the following:
American Ginseng: 12˜18 weight percent
Radix Astragali: 12˜18 weight percent
Rhizoma Polygonati Odorati: 12˜18 weight percent
Rhizoma Coptidis: 12˜18 weight percent
Radix Trichosanthis: 12˜18 weight percent
Radix Puerariae Lobatae: 12˜18 weight percent
Propolis: 2˜12 weight percent
Chromium: 0.01˜0.02 weight percent

Effects of ingredients are listed below:

American Ginseng: Increasing insulin production and decreasing cell apoptosis in pancreatic β-cells to lower blood sugar, increasing immunity, improving cardiac function, improving physical stamina and tiredness in people with chronic fatigue syndrome.(1)

Radix Astragali: Improving sugar metabolism to reduce blood sugar level, supporting kidney health by improving blood flow and laboratory markers of kidney function, maximizing heart health, protecting neurons, defending nerves from damage (2)

Rhizoma Polygonati Odorati: Lowering blood sugar, increasing immunity, improving heart function. (3)

Rhizoma Coptidis: Improving insulin sensitivity, increasing insulin secretion, and regulating carbohydrate metabolism (4)

Radix Trichosanthis: An abundant novel TK protein interacts with insulin receptor by docking analysis and activated the kinase activity of insulin receptor to lower the blood sugar. (5)

Radix Puerariae Lobatae: Stabilizing the blood sugar, improving cardiac function. (6)

Propolis: Improving insulin sensitivity to lower blood sugar level; effective in anticancer, antioxidant, anti-inflammatory, antibacterial, antifungal, and hypolipidemic (7)

chromium: Regulating of insulin action, metabolic syndrom, and cardiovascular disease. (8)

Indication of medication: Lower blood sugar, protect the kidney, and increas heart function. Reudce complications due to diabetes. Benefit the kidney and strengthen the heart. Tonify vital energy (Qi) and nourish Yin.

Z,999 provided herein.

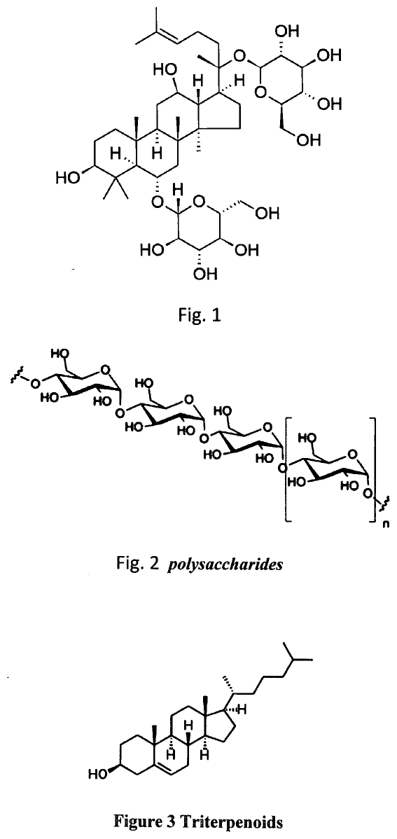

A dietary supplement composition provided herein includes Radix Panacis Quinquefolii extract. It mainly includes saponins, bolatile oils, amino acids, polyacetylenes, fatty acids, carbohydrates, sterols, inorganic elements, enzymes, flavonoids and so on. The main active ingredients are ginsenosides. Any appropriate amount of American Ginseng extract can be included within a dietary supplement composition provided in FIG. 1.

A dietary supplement composition provided herein includes Radix Astragali extract. It mainly includes polysaccharides, triterpenes and flavonoids. Any appropriate amount of Radix Astragali extract can be included within a dietary supplement composition provided in FIGS. 2 and 3.

A dietary supplement composition provided herein includes Rhizoma Polygonati Odorati. It mainly includes steroidal saponins, homoisoflavanones, volatile oils and polysaccharides. Any appropriate amount of Rhizoma Coptidis extract can be included within a dietary supplement composition provided herein.

A dietary supplement composition provided herein can include Rhizoma Coptidis. It mainly includes epiberberine, coptisine and jatrorrhizine. Any appropriate amount of Rhizoma Coptidis extract can be included within a dietary supplement composition provided herein.

A dietary supplement composition provided herein can include Radix Trichosanthis. It mainly includes trichosanthin, trichosanthin polysaccharide and trichosanthes kirilowii lectin. Any appropriate amount of Radix Trichosanthis extract can be included within a dietary supplement composition provided herein.

A dietary supplement composition provided herein can include Radix Puerariae Lobatae. It mainly includes puerosides and puerarin. Any appropriate amount of Radix Puerariae Lobatae extract can be included within a dietary supplement composition provided herein.

A dietary supplement composition provided herein can include Propolis. It mainly includes various flavonoids, aromatic acids, diterpenoid acids, triterpenoids and phenols. Any appropriate amount of Propolis can be included within a dietary supplement composition provided herein.

A dietary supplement composition provided herein can include one or more biologically active forms of chromium (Cr). The biologically active form of chromium can be a low-molecular-weight chromium-binding substance (LMWCr). A biologically active form of chromium can be naturally occurring oligopeptides. A dietary supplement composition provided herein can include chromium oligofructose complex which can also be called chromium amino acid polyfructose complex. A dietary supplement composition provided herein can also include chromium yeast, chromium picolinate, or chromium chelate. Any appropriate amount of chromium can be included within a dietary supplement composition provided herein.

A dietary supplement composition provided herein can be in the form of a beverage or a powder adapted to be mixed with water to form a beverage, liquid, soup, soup mix, solution, syrup, suspension, pill, powder, capsule, tablet, shake, bar, or other suitable ingestible carrier as is known in the art.

A dietary supplement composition provided herein can be prepared by conventional means with acceptable excipients such as binding agents, lubricants, fillers, thickeners, proteins, creamers, sweeteners, artificial sweeteners, disintegrants, or wetting agents. Liquid preparations for oral administration can take the form of soup, soup mix, solutions, syrups, or suspension, or they can be presented as a dry product for constitution with saline or other suitable liquid vehicle before use.

A dietary supplement composition provided herein also can contain acceptable additives as will be understood by one skilled in the art depending on the particular form of the dietary composition. Non-limiting examples of such additives include preservatives, coloring, flavoring, suspending agents, emulsifying agents, non-aqueous vehicles, buffer salts, and sweetening agents as appropriate. Non-limiting examples of specific additives include: water, beeswax, gelatin, glycerin, cocoa, caramel, carmine, lecithin, or titanium dioxide. Preparations for oral administration also can be suitably formulated to give controlled release of the ingredients.

A dietary supplement composition provided herein can contain an acceptable carrier for administration to a human or animal, including, without limitation, suspensions, sterile aqueous or non-aqueous solutions, and emulsions.

### Acute Toxicity Test

20 NIH mice (clean grade) weighing 20±2 g (4-6 week), randomly divided into two groups: ones in experiment group was orally administered with the present invention which is fully dissolved in 48 ml distilled water at its maximum tolerance dose of 0.4 ml/10 g three times a day. the other ones in the controlled group was treated with the same volume of distilled water.

Normal feeding was resumed 4 hours after the administration, continuous administration and observation last for 7 days. The death rate and its diet, activity, hair color and excretion were observed every day. 12 rats were randomly dissected after 7 days, 8 rats in the experiment group (4 of each sex) and 4 rats in the control group (2 of each sex). The internal effects on organs were studied.

No mice died and there were no significant changes in diet, activity, hair color and excretion within 7 days. After the dissection, the main organs of the rats in the experiment group were normal, which was no difference with the control group. The maximum tolerated dose (MTD) was considered to be 45 g/kg in this experiment which is 600 times more than the clinical dose. In conclusion, the dietary supplement composition provided herein is low in toxicity and safety.

Below is the detail of eight weeks' trial of the dietary supplement composition provided herein.

### Diagnostic, Inclusion, Exclusion Criteria

(1) Criteria for Diagnosing Diabetes Mellitus (DM)
By adopting suggested DM diagnosis and classification from criteria of consultant report and diagnosis of World Health Organization (WHO) 1999 (Department of Non-Communicable Disease Surveillance, WHO, Geneva Convention), using Vein Plasma Glucose (VPG) method:
Anyone with a condition matching the following may be diagnosed with DM:
1. With DM symptoms; two or more readings of Fasting Plasma Glucose (FPG)
   >7.0 mmol/L(126 mg/dl); With DM symptoms and Casual Plasma Glucose (CPG)
>11.1 mmol/L(200 mg/dl), or with Oral Glucose Tolerance Test (OGTT) 2-hour blood glucose≥11.1 mmol/L (200 mg/dl).
2. With no symptoms of DM, with FPG≥7.0 mmol/L or OGTT≥11.1 mmol/L or CPG ≥11.1 mmol/L, and with one more FPG≥7.0 mmol/L or OGTT≥11.1 mmol/L.

(2) Classification of DM
1. Type 1 DM: due to β-cell destruction, usually leading to absolute insulin deficiency.
   The characteristics are:
   1) Acute onset of the illness
   2) Typical case occurs in young child and adolescent, but may occur at any age.
   3) Low levels of plasma insulin and C peptides. Stimulation with sugar intake still results in low levels of secretion.
   4) Dependent on insulin treatment. Sudden removal of insulin will result in Diabetes Ketone Acidosis (DK) and is life-threatening.
   5) Inheritance is the major factor, expressed in variation of positive reaction rate of certain types of Human Leukocyte Antigen (HLA) on the 6th chromosome.
   6) The Islet Cell Antibody (ICA) presents positive test, especially at the initial stage.
2. Type 2 DM: due to a progressive insulin secretory defect on the background of insulin resistance. The characteristics are:
   1) Slower onset of the illness
   2) Typically seen in adults and seniors; occasionally in children.
   3) Plasma insulin level is just relatively low, with delayed release of insulin after sugar stimulation. Sometimes FPG value of obese patient tends to be higher, and insulin level after sugar stimulation is also higher than normal, but lower than that of non-DM patient of the same weight.
   4) Inheritance is an important trigger factor, but HLA test is negative.
   5) ICA negative test.
   6) Regularly poor response to insulin therapy
   7) Oral intake of anti-DM medication alone can control sugar level in general.
   There are obese and non-obese types based on weight.
3. Gestational DM: diabetes diagnosed in the second or third trimester of pregnancy that is not clearly overt diabetes.
4. Other Types of DM: due to other causes, e.g., monogenic diabetes syndromes (such as neonatal diabetes and maturity-onset diabetes of the young [MODY]), diseases of the exocrine pancreas (such as cystic fibrosis), and drug- or chemical-induced diabetes (such as in the treatment of HIV/AIDS or after organ transplantation)

(3) Traditional Chinese Medicine (TCM) Symptom Classification and Quantification Standard:

1. Thirst

| | |
|---|---|
| No symptom | point 0 |
| Water intake slightly more than usual | point 2 |
| Water intake increased 50% or more | point 4 |
| Water intake increased 100% or more | point 6 |

2. Fatigue

| | |
|---|---|
| No symptom | point 0 |
| Tires easily, able to perform physical | Point 2 |
| activity if insisted upon. | |
| Fatigue, but manages to perform daily activity | point 4 |
| Weakness of extremities, unable to support daily activity | point 6 |

3. Increased food intake with rapid onset of hunger

| | |
|---|---|
| No symptom | point 0 |
| Obvious hungry feeling | point 1 |
| Intolerable hungry feeling before meal | point 2 |
| Intolerable hungry feeling, commonly combined | point 3 |
| with low sugar reaction | |

4. Polyuria

| | |
|---|---|
| No symptom | point 0 |
| Slightly more amount of urine than usual, i.e., | point 1 |
| daily urine amount >2 liters | |
| Increased amount of urine, i.e., daily | point 2 |
| urine amount 2-3 liters | |
| Obvious increased amount of urine, i.e., daily | point 3 |
| urine amount >3 liters | |

5. Dry throat and dry mouth

| | |
|---|---|
| No symptom | point 0 |
| Slightly dry throat and mouth, alleviated by | point 1 |
| a small amount of water | |
| Dry throat and mouth, alleviated by drinking water | point 2 |
| Intolerable dry throat and mouth, unable to | point 3 |
| be alleviated by drinking water | |

6. Spontaneous sweating

| No symptom | | point 0 |
|---|---|---|
| Slight sweating after physical activity. Clothing | | point 1 |
| becomes faintly damp | | |
| Skin moistened without physical activity, | | point 2 |
| worsened by minor activity | | |
| | Commonly sweating, soaked from any physical activity | point 3 |

7. Night sweat

| | |
|---|---|
| No symptom | point 0 |
| Sweat occasionally on the head | point 1 |
| Sweat on chest and back, occurs repetitively | point 2 |
| Sweat all over the body, occurs frequently | point 3 |

8. Feverish sensation in the chest, palms and soles

| | |
|---|---|
| No symptom | point 0 |
| Feverish in the chest, palms and soles at night, | point 1 |
| with occasional dysphoria | |
| Feverish in palms and soles, with dysphoria often | point 2 |
| Highly feverish in palms and soles. | |
| Desires alleviation by holding cold items. | |
| Constant dysphoria and unsteadiness | point 3 |

(4) Inclusion Criteria
1. Age between 30 -70;
2. Meet criteria of DM syndrome diagnosis and the following two conditions:
   1) Type 2 Diabetes
   2) Has not been treated with diabetes medicine; already under diet control and kinesio therapy. After two weeks' stabilization, FPG between 7.8-13.9 mmol/L and 2-hour postprandial blood glucose (2-hour p.c.) test which is measuring blood glucose exactly 2 hours after eating a meal, times from the start of the meal, between 11.1-18.0mmol/L
   3) Has been treated with oral diabetic medicine; After two weeks' stabilization under diet control, kinesio therapy and oral diabetic medicine, FPG between 7.8-13.9 mmol/L and plasma glucose 2hr after meal between 11.1-18.0mmol/L
3. Meet criteria of TCM “Xiao Ke”, qi and yin deficiency by TCM differentiation of syndromes if the above-mentioned points are greater than 20.
4. Has DM medical history of more than 3 months
5. Has normal blood creatinine.
6. Woman of childbearing age must take effective birth control medications.
7. Has signed the informed consent form.

(5) Exclusion Criteria
1. Does not meet inclusion criteria
2. Has been affected by diabetic ketoacidosis and infection within a month.
3. With severe heart, liver, kidney, brain or blood system complications or combined with other severe primary illness, mental disease; with serum trans-ammonia enzyme level 1.5 times greater than normal.
4. Pregnancy or breast-feeding
5. Allergy to the test invention
6. Has recently participated in other clinical trials
7. Unable to cooperate, e.g. diet control or dosage compliance
8. Suspected or confirmed alcohol or drug abuse history.
9. Study researcher determines that the individual may not be able to fully participate due to pathological conditions or logistical reasons, e.g. working environment that could result in missing appointments.
10. Refuse to sign the agreement.

Participants and Study Design

(1) Treatment Group: 30 cases; Control Group: 30 cases.

(2) Course of Treatment: Eight weeks.

(3) Treatment and Diet Control
1. Pre-Experiment
Patients must be under strict dietary control and regular exercise for two weeks prior to the start of the experiment.
1) Test subjects without taking diabetes medications will not take any diabetes medicine.
2) Stop taking any diabetes dietary supplements.
3) Maintain the same diabetes medications and dosage without changing.
4) Test and record the blood glucose level after two weeks which should be stable.
The data will be the base before the experiment.

2. During the Experiment
1) All the blood glucose tests are taken before and after the treatment.
2) Do not take extra diabetes medicine or dietary supplements.
3) Do not change the diabetes medication or dosage.
4) Record the reason, time, frequency and medication for other disease treatment.
5) Gradually reduce the dosage or discontinuous the diabetes medication when the fasting plasma glucose (FPG) is less than 6.0 mmol/L and 2-hour postprandial blood glucose (2-hour p.c. blood glucose glucose) is less than 8.0 mmol/L) for two weeks. Closely monitor patient's vital signs and blood glucose level to adjust the dosage or medication accordingly.
6) Exclude the patient whose FPG is greater than or equals to 16.6mmo1/L and 2-hour p.c. blood glucose is greater than or equals to 22.2 mmol/L for one week after eliminating the predisposing factors.

3. Diet Control
1) Patients are required to maintain a consistent diet for an extended period of time.
2) Each patient's daily calorie intake is calculated based on his or her physical activities, body size and weight.
3) The diet contains 45-60% carbohydrates, less than 30% fat and protein which is based on 1 g/kg units in a day.
4) Total calories, especially those from carbohydrates, are distributed as ⅕, ⅖ and ⅖ for breakfast, lunch and dinner respectively.

(4) Observation
1. Background Check (before the test)
   1) Demographic Information: gender, age, height, weight, etc.
   2) General Clinical Information: medical history, illness condition, treatment history, complications and medications.
2. Safety Check
   1) Vital Signs: such as blood pressure, breath, heart beat rate. (before and after the test)
   2) Routine lab test of blood, urine and excrements. (before and after the test)
   3) Electrocardiogram, liver function test (ALT), renal function test (BUN, Cr) (Before and after the test)
   4) Adverse Events (document in detail anytime)
3. Therapeutic Effectiveness Check
   1) Major Indicators:
      Blood Glucose Test—FPG, 2-hour P.C. Blood Glucose (use venous blood plasma method. Take once each before and after treatment).
      TCM Symptoms Check (before the test, 4 weeks after medication, after the test).
   2) Secondary Indicators:
      Glycohemoglobin (HbAlc) (before and after the test).
      Blood Lipid includes Total Cholesterol (TC), Triglycerides (TG) (before and after the test)
      Symptoms Check (before the test, 4 weeks after medication, after the test)

(5) Clinical Trial
1. Clinic Visit 1: screening, inclusion period (day 0)
   This visit should complete the following tasks:
   1) Have test subjects to sign informed consent document
   2) Obtain medical history
   3) Obtain demography information
   4) Undergo total physical exam for vital physical signs
   5) Tests for FPG, OGTT 2-hour, glycol hemoglobin, blood lipid (TC, TG)
   6) Blood sample test for the safety and confirmation of the candidate's qualification
   7) TCM symptoms check
   8) Symptoms check
   9) Fill in disease baseline information
   10) Reevaluate inclusion/exclusion criteria
   11) Record current medication
   12) Distribute medication and complete the distribution record
2. Clinic Visit 2: trial period (two weeks after administering the medication)
   1) Inquire as to the illness condition, decide whether to continue or discontinue studying the subject.
   2) Check and count returned unused medication, decide whether to continue or discontinue the study on the test subject.
   3) Reduce or discontinue diabetes medication.
   4) Inquire or record concomitant medication and adverse events.
   5) Test blood glucose with FPG and 2-hour p.c. blood glucose.
3. Clinic Visit 3: trial period (four weeks after medication administering)
   1) Inquire as to the illness condition, decide whether to continue or discontinue studying the subject.
   2) Check and count returned unused medication, decide whether to continue or discontinue the study on the test subject.
   3) Record clinical symptoms.
   4) Document concomitant medications and adverse events
   5) Blood taken from the fingertips for FPG and 2-hour p.c. blood glucose test.
   6) Collect blood samples for laboratory to test for safety
   7) Reduce or discontinue diabetes medication.
   8) TCM symptoms check
   9) Symptoms Check
   10) Document conditions of either reducing the dosage or discontinuing diabetes medications.
   11) Document concomitant medications or adverse events
   12) Fill out the record form for returned medication
4. Clinic Visit 4: trial period (six weeks after medication administering)
   1) Inquire as to the illness condition, decide whether to continue or discontinue studying the subject.
   2) Check and count returned unused medication, decide whether to continue or discontinue the study on the test subject.
   3) Record clinical symptoms.
   4) Document concomitant medications and adverse events
   5) Blood taken from the fingertips for FPG and 2-hour p.c. blood glucose test.
   6) Collect blood samples for laboratory to test for safety
   7) Reduce or discontinue diabetes medication.
   8) Document conditions of either reducing the dosage or discontinuing diabetes medications.
   9) Document concomitant medications or adverse events
   10) Fill out the record form for returned medication
5. Clinic Visit 5: end of trial period (eight weeks after medication administering)

### Results

1. Primary Criterion of Curative Effect Evaluating
1. FPG Test:
   Obviously Effective: after the treatment, FPG<7.0 mmol/L, or there is more than 30% decreasing on FPG.
   Effective: after the treatment, FPG is 10% lower or more than it before the treatment.
   No Effect: after the treatment, FPG is not lowered to the criterion above.

| | | | | | Markedly | Total |
|---|---|---|---|---|---|---|
| | | Obviously | | No | Effective | Effective |
| Group | n | Effective | Effective | Effect | Rate | Rate |
| Treatment | 30 | 16 | 12 | 2 | 53.33% | 93.33%* |
| Group | | | | | | |
| Control | 30 | 1 | 2 | 27 | 3.33% | 10% |
| Group | | | | | | |

*Compare to the Control Group, P < 0.05

2. 2-hour P.C. Blood Glucose Test:
   Obviously Effective: after the treatment, 2-hour p.c. blood glucose<7.8 mmol/L, or has been reduced to more than 30% of the level before the treatment.
   Effective: after the treatment, 2-hour p.c. blood glucose has been reduced to more than 10% of the level before the treatment.
   No Effect: after the treatment, 2-hour p.c. blood glucose is not lowered to the criteria level above.

| | | | | | Markedly | Total |
|---|---|---|---|---|---|---|
| | | Obviously | | No | Effective | Effective |
| Group | n | Effective | Effective | Effect | Rate | Rate |
| Treatment | 30 | 18 | 11 | 1 | 60% | 96.67%* |
| Group | | | | | | |
| Control | 30 | 0 | 1 | 29 | 0 | 3.33% |
| Group | | | | | | |

*Compare to the Control Group, P < 0.05

3. TCM Symptoms Evaluation:
   Evaluating the curative effect on TCM symptoms on the basis of RATE calculation.
   Clinical Control: TCM clinical symptoms—disappearing of physical signs or basic signs, RATE≥95%.
   Obviously Effective: 70%≤RATE<95%
   Effective: 30%+RATE<70%
   No Effect: RATE<30%
   RATE=[(points before the treatment−points after the treatment)/points before the treatment]×100%.

| | | | | | Markedly | Total |
|---|---|---|---|---|---|---|
| | | Obviously | | No | Effective | Effective |
| Group | n | Effective | Effective | Effect | Rate | Rate |
| Treatment | 30 | 18 | 11 | 1 | 60% | 96.67%* |
| Group | | | | | | |
| Control | 30 | 0 | 1 | 29 | 0 | 3.33% |
| Group | | | | | | |

*Compare to the Control Group, P < 0.05

2. Secondary Criterion of Curative Effect Evaluation
1. Glycohemoglobin (HbAlc):
   Evaluate with X±SD, where the data are based on the glycohemoglobin before and after the treatment.

| | | Before | After |
|---|---|---|---|
| Group | n | Treatment % | Treatment % |
| Treatment Group | 30 | 6.5 ± 1.23 | 5.23 ± 0.98^{Δ} * |
| Control Group | 30 | 6.41 ± 2.34 | 6.58 ± 1.56 |

^{Δ}Compare to Before Treatment, P < 0.05;
* Compare to Control Group, P < 0.05

2. Total Cholesterol:
   Evaluate with X±SD, where the data are based on the total cholesterol before and after the treatment.

| | | Before Treatment | After Treatment |
|---|---|---|---|
| Group | n | mmol/L | mmol/L |
| Treatment Group | 30 | 5.67 ± 1.13 | 4.39 ± 1.21^{Δ} * |
| Control Group | 30 | 5.71 ± 2.01 | 5.76 ± 1.16 |

^{Δ}Compare to the Before Treatment, P < 0.05;
* Compare to the Control Group, P < 0.05

3. Triglycerides (TG):
   Evaluate with X±SD, where the data are based on the TG before and after the treatment.

| | | Before Treatment | After Treatment |
|---|---|---|---|
| Group | n | mmol/L | mmol/L |
| Treatment Group | 30 | 2.45 ± 0.53 | 1.65 ± 0.98^{Δ} * |
| Control Group | 30 | 2.41 ± 0.44 | 2.55 ± 0.76 |

^{Δ}Compare to previous Treatment, P <0.05;
* Compare to the Control Group, P <0.05.

4. Symptoms:
   Vanished: the symptom is gone
   Improved: the points decreased, but the symptom hasn't disappeared completely.
   No Change: the points has no change after the treatment
   Exacerbated: the points increased after the treatment

| | | | | | | Total |
|---|---|---|---|---|---|---|
| | | | Im- | No | Exacer- | Effec- |
| Group | n | Vanished | proved | Change | bated | tive |
| Treatment | 30 | 11 | 10 | 9 | 0 | 70%* |
| Group | | | | | | |
| Control | 30 | 1 | 3 | 20 | 6 | 13.33% |
| Group | | | | | | |

*Compare to the Control Group, P < 0.05.

3. No adverse event occurred.

Though the invention has been described with respect to specific preferred and alternative embodiments, many additional variations and modifications will become apparent to those skilled in the art upon reading the present application. Thus, it is the intention that the appended claims be interpreted as broadly as possible in view of the prior art to include all such variations and modifications.

We claim:

1. An herbal dietary supplement comprising essentially of: a) between 12 and 18 weight percent of American Ginseng extract; b) between 12 weight percent and 18 weight percent of Radix Astragali extract; c) between 12 weight percent and 18 weight percent of Rhizoma Polygonati Odorati extract; e) and an extract or extracts selected from the group consisting of : i: between 12 weight percent and 18 weight percent of Rhizoma Coptidis extract, ii:
between 12 weight percent and 18 weight percent of Radix Trichosanthis extract, iii: between 12 weight percent and 18 weight percent of Radix Puerariae Lobatae extract; iv: between 2 weight percent and 12 weight percent of Propolis; v: between 0.01 and 0.02 weight percent of chromium; vi: and combinations thereof.

2. The herbal dietary supplement composition of claim 1, wherein said American Ginseng extract is in an amount of between 2 mg and 15000 mg.

3. The herbal dietary supplement composition of claim 1, wherein said Radix Astragali extract is in an amount of between 2 mg and 15000 mg.

4. The herbal dietary supplement composition of claim 1, wherein said Rhizoma Polygonati Odorati extract is in an amount of between 2 mg and 15000 mg.

5. The powder composition of claim 1, wherein the composition further consists of an extract or extracts selected from the group consisting of a: between 12 weight percent and 18 weight percent of Rhizoma Coptidis extract, b: between 12 weight percent and 18 weight percent of Radix Trichosanthis extract, c: between 12 weight percent and 18 weight percent of Radix Puerariae Lobatae extract; d: between 2 weight percent and 12 weight percent of Propolis; e: between 0.01 and 0.02 weight percent of chromium; f: and combinations thereof.

6. The herbal dietary supplement composition of claim 5, wherein said Rhizoma Coptidis extract is in an amount of between 2 mg and 15000 mg.

7. The herbal dietary supplement composition of claim 5, wherein said Radix Trichosanthis extract is in an amount of between 2 mg and 15000 mg.

8. The herbal dietary supplement composition of claim 5, wherein said Radix Puerariae Lobatae extract is in an amount of between 2 mg and 15000 mg.

9. The herbal dietary supplement composition of claim 5, wherein said Propolis is in an amount of between 20 mg and 1000 mg.

10. The herbal dietary supplement composition of claim 5, wherein said chromium is in an amount of between 0.2 mcg and 1000 mcg.

11. The herbal dietary supplement composition of claim 1, wherein said amount contains less than 5 grams of sugars.

12. The herbal dietary supplement composition of claim 1, wherein the herbal dietary supplement composition is substantially free of hydrogenated or trans fats.

13. The herbal dietary supplement composition of claim 1, wherein said composition further consists essentially of an artificial sweetener which is selected from the group consisting of acesulfame potassium, aspartame, saccharine, sucralose, and combinations thereof.

14. The herbal dietary supplement composition of claim 1, wherein said composition further consists essentially of a flavorant selected from the group consisting of artificial vanilla flavorant, vanilla extract, artificial chocolate flavorant, caramel, cocoa powder, cream, dried cream extracts, and combinations thereof.

15. The herbal dietary supplement composition of claim 1, wherein said composition further consists essentially of a component selected from the group consisting of agar, alginates, dextrin, calcium caseinate, carboxymethyl cellulose, carrageenan, casein, chitin, collagen, hydroxypropyl cellulose, hydroxyethyl cellulose, hydroxypropyl methyl cellulose, karaya, konjac, methyl cellulose, wheat gluten, propylene glycol alginate, xanthan, dextrin, pullulan, curdlan, gellan, locust bean gum, inulin, guar gum, tara gum, gum tragacanth, pectin, gelatin, psyllium seed, chitosan, gum acacia, polyvinyl pyrrolidone, polyethylene oxide, polyvinyl alcohol, zein, and combinations thereof.

16. The herbal dietary supplement composition of claim 15, wherein said composition further consists of inulin, wherein the inulin makes up no more than 15 weight percent of the herbal dietary supplement composition.

17. The herbal dietary supplement composition of claim 16, wherein the inulin makes up between 5 and 15 weight percent of the herbal dietary supplement composition.

18. The herbal dietary supplement composition of claim 16, wherein the inulin makes up between 0 and 5 weight percent of the herbal dietary supplement composition.

19. The herbal dietary supplement composition of claim 15, wherein said composition further consists of calcium caseinate, wherein the calcium caseinate makes up between 5 and 7 weight percent of the herbal dietary supplement composition.

20. The herbal dietary supplement composition of claim 15, wherein said composition further consists of salt, wherein the salt makes up between 0 and 2 weight percent of the herbal dietary supplement composition.

21. The herbal dietary supplement composition of claim 1, wherein the herbal dietary supplement composition is substantially free of corn syrup.

22. The herbal dietary supplement composition of claim 1, wherein said protein contains between 10 and 13 weight percent soy protein, between 8 and 12 percent whey protein concentrate, and between 5 and 8 weight percent whey protein isolate.

23. The herbal dietary supplement composition of claim 1, wherein the powdered dietary supplement composition is substantially free of vitamins or mineral fortification other than the chromium.

24. A tablet, capsule, liquid, beverage, soup, soup mix, solution, syrup, suspension, pill, powder, shake, bar consisting essentially of an American Ginseng extract, Radix Astragali extract, and Rhizoma Polygonati Odorati extract.

25. A tablet, capsule, liquid, beverage, soup, soup mix, solution, syrup, suspension, pill, powder, shake, bar consisting essentially of an American Ginseng extract, Radix Astragali extract, Rhizoma Polygonati Odorati extract, and an extract or extracts selected from the group consisting of Rhizoma Coptidis extract, Radix Trichosanthis extract, Radix Puerariae Lobatae extract, Propolis, chromium, and combinations thereof.

26. The tablet, capsule, liquid, beverage, soup, soup mix, solution, syrup, suspension, pill, powder, shake, bar of claim 25, wherein the composition further consists of an extract or extracts selected from the group consisting of: a: between 2 mg and 15000 mg of Rhizoma Coptidis extracts; b: between 2 mg and 15000 mg of Radix Trichosanthis extracts; c: between 2 mg and 15000 mg of Radix Puerariae Lobatae extracts; d: between 2 weight percent and 12 weight percent of Propolis; e: between 0.01 and 0.02 weight percent of chromium; and f: combinations thereof.

27. The tablet, capsule, liquid, beverage soup, soup mix, solution, syrup, suspension, pill, powder, shake, bar of claim 26, wherein said tablet, capsule, liquid or beverage contains Rhizoma Coptidis extract in an amount of between 2 mg and 15000 mg.

28. The tablet, capsule, liquid, beverage, soup, soup mix, solution, syrup, suspension, pill, powder, shake, bar of claim 26, wherein said tablet, capsule, liquid or beverage contains Radix Trichosanthis extract in an amount of between 2 mg and 15000 mg.

29. The tablet, capsule, liquid, beverage, soup, soup mix, solution, syrup, suspension, pill, powder, shake, bar of claim 26, wherein said tablet, capsule, liquid or beverage contains Radix Puerariae Lobatae extract in an amount of between 2 mg and 15000 mg.

30. The tablet, capsule, liquid, beverage, soup, soup mix, solution, syrup, suspension, pill, powder, shake, bar of claim 26, wherein said Propolis is in an amount of between 20 mg and 1000 mg.

31. The tablet, capsule, liquid, beverage, soup, soup mix, solution, syrup, suspension, pill, powder, shake, bar of claim 26, wherein said chromium is in an amount of between 0.2 mcg and 1000 mcg.
